(19) United States
(12) Patent Application Publication
(10) Pub. No.: US 2004/0121377 A1
(43) Pub. Date: Jun. 24, 2004

(54) Spatially encoded and mobile arrays of tethered lipids

(76) Inventors: Chiaki Ishii, Menlo Park, CA (US); Steven G. Boxer, Stanford, CA (US); Lance Kam, New York, NY (US)

Correspondence Address: PERKINS COIE LLP, P.O. BOX 2168, MENLO PARK, CA, 94026, US

(21) Appl. No.: 10/680,356

(22) Filed: Oct. 6, 2003

Related U.S. Application Data
(60) Provisional application No. 60/416,066, filed on Oct. 5, 2002.
(60) Provisional application No. 60/416,065, filed on Oct. 5, 2002.

(51) Int. Cl. C12Q 1/68; C12M 1/34

(52) U.S. Cl. 435/6; 435/287.2

(57) ABSTRACT

Arrays of separated lipid bilayers are disclosed. Also disclosed are methods of using the arrays and methods of forming the arrrays. In addition, the invention provides useful systems and methods for selectively removing, collecting, and reconstitute lipid bilayers from specified regions of a surface. A stream of detergent solution is directed over a preformed bilayer, resulting in the removal of bilayer material. Diffusion-limited mixing between adjacent flows at low Reynolds number provides confinement of this stripping solution and, consequently, high precision. The freshly exposed surface allows formation of new connected bilayer when exposed to lipid vesicles. In conjunction with surface micropatterning and electrophoretic manipulation, the invention also provides a membrane-based separation/purification strategy.

### CROSS REFERENCE TO RELATED APPLICATIONS

This application claims priority to U.S. Provisional Patent Application Nos. 60/416,065 and 60/416,066, both of which were filed on Oct. 5, 2002 and both of which are incorporated herein in their entireties by reference.

### FIELD OF THE INVENTION

The present invention relates to arrays of separated lipid bilayers and to methods of making and using such array. The invention also relates to methods for manipulating lipid-bilayer regions on a substrate, and to systems for using such methods.

### BACKGROUND OF THE INVENTION

Lipid vesicles are widely used as mimics for cell membranes. Many cellular components including a large number of potential drug targets are associated with cell membranes. Lipid bilayers on solid supports are especially challenging because they are two-dimensional fluids. Methods for patterning and displaying membrane components such as lipids and some membrane tethered or anchored proteins have been described on patterned supported bilayers. Often these methods fail for membrane tethered proteins and they nearly always fail for integral membrane proteins.

This failure is believed to be caused by interactions between regions of the membrane protein that are outside the lipid milieu and therefore interact strongly with the glass surface. This can cause denaturation of the protein with loss of function or it can limit the lateral mobility of the membrane protein, often important for function. Some investigators have described strategies for lifting the supported bilayer away from the underlying solid support by the use of polymer cushions or by some tethering strategies. In most cases, a substantial immobile fraction of proteins is observed and the activity of the proteins in such cushioned bilayers is often reduced.

Biomolecules in supported lipid bilayers of appropriate composition freely diffuse within the plane of the membrane, mimicking a property of cellular membranes that is essential for many cellular functions⁴⁻⁸. The compositions of supported lipid bilayers are easily controlled, providing a robust model of systems ranging from membrane-associated biomolecules (e.g., integrins, gap junctions, ion channels, GPI-anchored proteins, and synthetic peptides) to cells of the immune system⁷⁻¹¹.

In recent years, a number of tools have been introduced for dynamically manipulating pre-formed supported lipid bilayers, leading to a variety of novel, bilayer-based systems and new models for examining membrane biomolecules^{12,13}. Also see, e.g., copending U.S. patent application Ser. No. 09/680,637, filed Oct. 6, 2000; Attorney Docket No. 58600-8204.US00, filed May 17, 2001; U.S. Pat. No. 6,228,326, issued May 8, 2001, and U.S. Patent Application Publication No. 2002/0009807 A1, published Jan. 24, 2003, each of which is incorporated by reference herein in its entirety.

Two core concepts are membrane micropatterning or corralling, and membrane electrophoresis. In membrane micropatterning, barrier materials are patterned onto glass or other appropriate surfaces using standard microfabrication or microcontact printing methods¹⁴⁻¹⁶. When vesicles from solution contact the surface they irreversibly stick and ultimately form a continuous bilayer on the open glass regions, but not on the barrier materials; the barriers restrict motion of lipids to specific regions of the surface. One use of these barriers is to create multiple, isolated lipid bilayer patches or corrals, on a substrate. FIG. 13A illustrates schematically one such corral containing biomolecules of interest, which, for simplicity here, are lipids marked either red or green that are negatively charged or neutral, respectively. Lipids are free to diffuse within the corralled regions but do not cross between neighboring corrals; in the absence of any driving force or strong intermolecular associations, the bilayer composition is uniform across the corralled region. In membrane electrophoresis, an electric field parallel to the plane of the bilayer induces a net drift of charged membrane-associated components. When confined by micropatterened barriers, this drift, in competition with 2-dimensional diffusion, leads to the formation of gradients of the charged components in the vicinity of the barrier¹⁷. This process is illustrated in FIG. 13B by the accumulation of the charged lipids (red), but not the neutral components (green), against the right-hand barrier.

Two other concepts in the manipulation of lipid bilayers also exist. First, it is possible to remove sections of a bilayer from a surface either by mechanically scratching the surface^{17,18} or by blotting with a polymer stamp^{19,20}. The remaining supported bilayer can expand into the region where membrane was removed; however, the expansion is self-limiting²¹ and so long as a sufficient area is removed, the gap does not heal over^{19,21}. Such gaps can be completely healed by reintroducing fresh vesicles¹⁸. The second concept exploits both the mechanism of supported bilayer formation and patterning. In bilayer formation, the initial contact and sticking of vesicles to a surface is irreversible. As a consequence, whatever composition of vesicles approaches the surface is captured by the surface. By using adjacent flows of vesicle solutions with different compositions in a laminar flow configuration, mixing is diffusion limited. Limited mixing of two solutions containing vesicles with different compositions has been obtained²²; the relative concentration of these two types of vesicles in solution varied gradually and continuously over a transitional mixing region at the interface between the two flows. This process yields a bilayer of different composition at different positions on the surface within the mixing region. If the surface is not patterned, then the bilayer components mix and the initial variations in composition disappear. However, by patterning the surface with corrals of small extent compared to the width of the mixing region, the mixing of vesicle composition is limited locally, and this leads to formation of composition arrays. More generally, patterning a surface with features of small extent compared to the spatial variations in vesicle composition allows capture of these variations in the resultant bilayer.

It would therefore be desirable to provide a method for capturing the advantages of spatial organization and lateral mobility that is present in a supported bilayer, while retaining function as found in vesicles. It would also be highly desirable to provide a method that allows removal of a bilayer from a specific area on a surface using laminar flow, and in some cases, replacement of this with a bilayer of a different composition or a barrier material. It would also be desirable to provide a method of directed removal/replacement of lipid bilayers using regions of extent much smaller than the size of the corrals. The present invention is designed to meet these needs.

### SUMMARY OF THE INVENTION

The invention provides, in one aspect, an array of separated lipid bilayers. The array includes a substrate having a surface defining a plurality of distinct bilayer-compatible surface regions. Also included are a plurality of discrete lipid bilayer expanses in associated surface regions, said expanses having inner and outer bilayer surfaces, and an aqueous film interposed between each bilayer-compatible surface region and the lower surface of the corresponding lipid bilayer expanse. Each of the expanses contains one or more lipids derivatized with an oligonucleotide having a patch-specific oligonucleotide sequence and extending from the outer surface of the associated expanse. A bulk aqueous phase covers the lipid bilayer expanses. At least one biomolecule is anchored to at least one of the lipid bilayer expanses through a complementary oligonucleotide sequence capable of specifically hybridizing with the patch-specific oligonucleotide sequence in that expanse, such that the biomolecule is anchored to that expanse.

In one embodiment of the invention, the array also includes one more discrete lipid bilayer patches associated with the expanses, where each such patch contains such a biomolecule anchored to the associated expanse through said hybridized oligonucleotides. In a related embodiment, the lipid bilayer patches on different associated expanses have different compositions. The different compositions of each lipid bilayer patch may be encoded by the patch-specific oligonucleotide sequence in the expanse.

In another embodiment of the invention, one or more of the lipid bilayer patch is a vesicle.

In yet another embodiment, the array also includes one or more second biomolecules associated with the bilayer patches, the second biomolecule(s) being able to move substantially freely within the associated patch. At least some of the different bilayer patches may have different second biomolecules.

In one embodiment, the biomolecule corresponds to the oligonucleotide sequence, such that the identity of the biomolecule may be determined from the sequence of the oligonucleotide.

In yet another embodiment, the discrete lipid bilayer expanses in associated surface regions are separated by one or more barrier regions. Alternatively, the discrete lipid bilayer expanses in associated surface regions are separated from one another by self-limiting lateral diffusion, without physical barriers between the expanses on the substrate surface.

In yet, still another embodiment, the distinct bilayer-compatible surface regions on the substrate are formed from a material selected from the group consisting of SiO₂, MgF₂, CaF₂, and mica.

In another embodiment, the lipid bilayer expanses are comprised of at least one lipid selected from the group consisting of phosphatidylcholine, phosphatidylethanolamine, phosphatidylserine, phosphatidic acid, phosphatidylinositol, phosphatidylglycerol, and sphingomyelin.

Another aspect of the invention includes a method of using a lipid patch array to detect membrane-bound biomolecular interactions. The method includes incubating any of the arrays described above under conditions effective to allow for the formation of biomolecular complexes between the second biomolecules, and detecting any formed biomolecule complexes.

In one embodiment, the method is used screening for molecules that enhance or disrupt membrane-bound biomolecular interactions. In this embodiment, the method further includes contacting the array, prior to or after said incubating, with one or more molecules under conditions which allow for the interaction of said molecules with said biomolecules or biomolecular complexes, detecting any formed biomolecular complexes, and comparing the results from the previous step to the results from the detecting step of the method described above to determine whether the one or more molecules enhanced or disrupted membrane-bound biomolecular interactions. The degree of complex formation may be quantitated.

The biomolecules may be selected from the group consisting of peptides, proteins, carbohydrates, cytokines, growth factors, hormones, enzymes, toxins, drugs, oligonucleotides, lipids, and combinations thereof. The molecules may be selected from the group consisting of peptides, proteins, carbohydrates, cytokines, growth factors, hormones, enzymes, toxins, drugs, oligonucleotides, lipids, and combinations thereof.

In another embodiment, a method of manipulating lipid-bilayer regions on a substrate is contemplated. The method includes applying, to the array described above, a controlled laminar-flow stream of an aqueous liquid, under flow conditions effective to remove a portion of the expanse in the path of said stream, wherein remaining portions of said expanse are substantially retained in their original position(s) on said region, adjacent exposed portion(s) of said region.

It is another object of the invention to provide a method of manipulating lipid-bilayer regions on a substrate. The method includes the steps of applying, to a substrate having formed thereon, a discrete, defined-shaped lipid expanse which is confined to a corresponding defined-shaped region on the substrate and separated by an aqueous film, a controlled laminar-flow stream of an aqueous liquid. The stream is applied under flow conditions effective to remove a portion of the expanse in the path of the stream. The remaining portions of said expanse are substantially retained in their original position(s) on the region, adjacent exposed portion(s) of the region.

In one embodiment, the discrete lipid bilayer expanse further includes one or more biomolecules in the expanse. The biomolecules may be selected from the group consisting of peptides, proteins, carbohydrates, cytokines, growth factors, hormones, enzymes, toxins, drugs, oligonucleotides, lipids, and combinations thereof.

The method may also include, in one embodiment of the invention, applying to the exposed portion(s) of the region, one or more lipid bilayer patches, such that the lipid bilayer patch(es) replaces the removed portion of the lipid bilayer expanse. In a related embodiment, the discrete, defined-shaped lipid expanse further includes one or more first biomolecules in the expanse, and/or the lipid bilayer patch further includes one or more second biomolecules in the patch(es), such that the first and/or second biomolecules are capable of moving between the expanse and the adjacent patch(es).

In another embodiment, the method includes applying to the exposed portion(s) of the region, one or more barrier regions, such that the barrier region(s) replaces the removed portion of the lipid bilayer expanse. In one embodiment, the barrier region includes a protein.

The above embodiments of the method of the invention may also include inducing a driving force such that the biomolecules in the discrete, defined-shaped lipid expanse are positioned in a desired location(s) within the expanse. The driving force may be induced prior to, and/or following, the applying step described above. In one embodiment, the driving force is an electric field.

In yet another embodiement of the invention, the removed portion(s) of the lipid bilayer expanse are collected for further analysis. Such analysis may include capillary electrophoresis, mass spectrometry and enzyme-based colorimetry.

The aqueous liquid in the embodiments of the methods described above may include a detergent. In one embodiment, the aqueous liquid includes between about 0.1% to about 5% w/v detergent. Preferably, the aqueous liquid comprises between about 1% to about 2% w/v detergent. In a related embodiment, the detergent is n-octyl-β-D-glucopyranoside.

In one embodiment of the invention, the removed portion of the expanse is between about 0.1 μm and about 100 μm in width. Preferably, the removed portion is between about 1 μm and about 10 μm in width.

In yet another embodiment, the method is used for producing a selected pattern on the surface of the substrate. In this embodiment, the method also includes a plurality of controlled laminar flow streams of one or more aqueous liquids which are applied under flow conditions effective to remove a plurality of portions of the expanse in the path of the streams.

Another aspect of the present invention includes a system for manipulating lipid-bilayer regions on a substrate. The system includes the substrate having formed thereon, a discrete, defined-shaped lipid expanse which is confined to a corresponding defined-shaped region on the substrate and separated by an aqueous film. The system also includes a flow generating device capable of applying a controlled laminar-flow stream to the substrate and a composition capable of selectively removing at least a portion of the expanse. Preferably, the composition is an aqueous liquid as described above.

In one embodiment, the defined-shaped region on the substrate is formed from a material selected from the group consisting of SiO₂, MgF₂, CaF₂, and mica.

The lipid expanse may include at least one lipid selected from the group consisting of phosphatidylcholine, phosphatidylethanolamine, phosphatidylserine, phosphatidic acid, phosphatidylinositol, phosphatidylglycerol, and sphingomyelin.

In another embodiment, the system includes one or more biomolecules in the discrete lipid bilayer expanse.

These and other objects and features of the invention will be more fully appreciated when the following detailed description of the invention is read in conjunction with the accompanying figures.

### BRIEF DESCRIPTION OF THE FIGURES

FIG. 1 depicts a supported lipid bilayer on a substrate according to one embodiment of the invention;

FIG. 2 shows a patterned supported lipid bilayer. The barriers define regions on the surface where the bilayer assembles and the barriers corral the bilayer to specific regions on the surface. The bilayer is fluid within each corral.

FIG. 3 is a schematic diagram of a lipid vesicle, cut away to show the interior and exterior surfaces. The composition of lipids, inserted proteins and contents of vesicles may be varied.

FIG. 4 illustrates two methods for the synthesis of oligolipids in which an oligonucleotide is covalently attached to a lipid head group. The oligonucleotide sequence R′ is specified by synthesis and the lipid side chains may be varied.

FIG. 5 depicts oligolipids with sequence A′ displayed on a fluid supported bilayer surface. The oligolipids, like the lipids, are free to diffuse laterally in the plane of the bilayer.

FIG. 6 shows two different sequences of oligolipids, A′ and B′, displayed in different regions of a patterned fluid according to one embodiment of the invention;

FIG. 7 depicts a vesicle displaying oligolipid A, whose sequence is complementary to A′ which is displayed on the supported bilayer surface, and becomes tethered to the surface by sequence recognition and double helix formation. The tethered vesicle is laterally mobile in the plane parallel to the bilayer surface.

FIG. 8 illustrates the encoded tethering of different vesicles, e.g. differing in content, associated proteins, etc. A vesicle displaying oligolipid A, whose sequence is complimentary to A′ which is displayed on the supported bilayer surface, becomes tethered to the surface by sequence recognition and double helix formation. A vesicle displaying oligoipid B, whose sequence is complimentary to B′ which is displayed on the supported bilayer surface, becomes tethered to the surface by sequence recognition and double helix formation. This places vesicles whose contents are encoded by oligolipid B in the corralled region on the right.

FIG. 9 shows epifluorescence images of single vesicles tethered to a supported lipid bilayer. The tethered vesicles were labeled with a lipid bearing a fluorescent label for visualization. Individual spots are individual tethered vesicles. The image is partitioned by a barrier and the trajectories taken over time of individual tethered vesicles.

FIG. 10 illustrates encoded tethering of vesicles displaying different membrane proteins. A vesicle displaying oligolipid A, whose sequence is complimentary to A′ which is displayed on the supported bilayer surface, becomes tethered to the surface by sequence recognition and double helix formation. A vesicle displaying oligolipid B, whose sequence is complimentary to B′ which is displayed on the supported bilayer surface, becomes tethered to the surface by sequence recognition and double helix formation. This places vesicles whose contents is encoded by oligolipid B in the corralled region on the right.

FIG. 11 depicts the interactions between vesicles, proteins on their surface and/or vesicle content can occur when tethered vesicles diffusing on the surface encounter. This process can be enhanced or disrupted by agents in solution or associated with the vesicles.

FIG. 12 shows the formation of a tethered bilayer by oligolipid complimentarity. A large vesicle or vesicles displaying oligolipid A and containing a specific protein and/or lipid composition is bound to a supported lipid bilayer displaying the complimentary oligolipid A′. Upon rupture, for example by osmotic stress, a bilayer patch becomes tethered to the supported bilayer. The distance of separation is determined by the length of the oligonucleotide sequence. The diagram also illustrates the tethering different bilayer patches to different regions on a supported lipid bilayer surface by using the encoded vesicle tethering method described herein.

FIGS. 13A-13D illustrate the steps in the method of micropatterning, electrophoresis, and stripping of lipid bilayer regions on a substrate in accordance with one embodiment of the invention. (13A) Schematic diagram of a supported lipid bilayer confined or corralled by microfabricated barriers. Mobile species, illustrated by lipids with red and green head groups, freely diffuse and mix, and approach a uniform concentration across the extent of lipid bilayer. (13B) Application of an electric field induces migration of charged membrane components (the red lipids), and represents one method of manipulating a lipid bilayer after formation; the neutral lipids (green) do not respond to this applied field and remain homogeneous in the corral. (13C) A stream of stripping solution is flowed over part of the surface under laminar flow conditions leaving an open region on the substrate. (13D) A new lipid bilayer, which could contain new biomolecular species, indicated by the lipid with blue headgroups, is introduced.

FIGS. 14A-14F illustrate the preparation of purified populations of mobile membrane biomolecules according to another embodiment of the invention. (14A) A laminar flow system is used to prepare two connected regions of lipid bilayer. The one on the left was formed from vesicles containing a target biomolecule, which yields both mobile and immobile target biomolecules, indicated by the green, upright “Y” and membrane-embedded forms, respectively. The region on the right was formed from pure vesicles not containing the target biomolecule. (14B) Under the influence of an electric field or by simple diffusion, the mobile biomolecules, but not the immobile counterparts, move into the region initially composed of bilayer alone; this region now contains a purified population of mobile target biomolecules. (14C) Diffusion of mobile target biomolecules back to the left can be prevented by removing a thin strip of bilayer from the middle of the sample, which (14D) can be filled in with new material to form a permanent barrier. (14E) Alternatively, the region containing the immobile biomolecules on the left may be completely removed. (14F) Subsequently, either more of the target biomolecule or a new biomolecular species (shown in red) could be introduced into this system in either a single or multiple steps. This process ensures that the various biomolecules are mobile.

FIGS. 15A-15C illustrate spatially-selective removal of molecules from the surface for compositional analysis. (15A) Application of an electric field to a corralled region with several components (shown in green, blue and red) leads to reorganization depending on their size, charge, and mutual interaction(s). Although visualization by fluorescence microscopy can be very useful, there are many situations in which it would be desirable to probe the composition with spatial selectivity but without labeling. (15B) Under laminar flow conditions, application of several separate streams of stripping solution captures the distribution of biomolecules in A and delivers the contents to separate containers for subsequent analysis, e.g. by mass spectrometry. (15C) Alternatively, a single stream of stripping solution can be scanned across the sample, sequentially removing regions of the bilayer for collection and analysis.

### DETAILED DESCRIPTION OF THE INVENTION

I. Definitions

Unless otherwise indicated, all technical and scientific terms used herein have the same meaning as they would to one skilled in the art of the present invention. It is to be understood that this invention is not limited to the particular methodology, protocols, and reagents described, as these may vary.

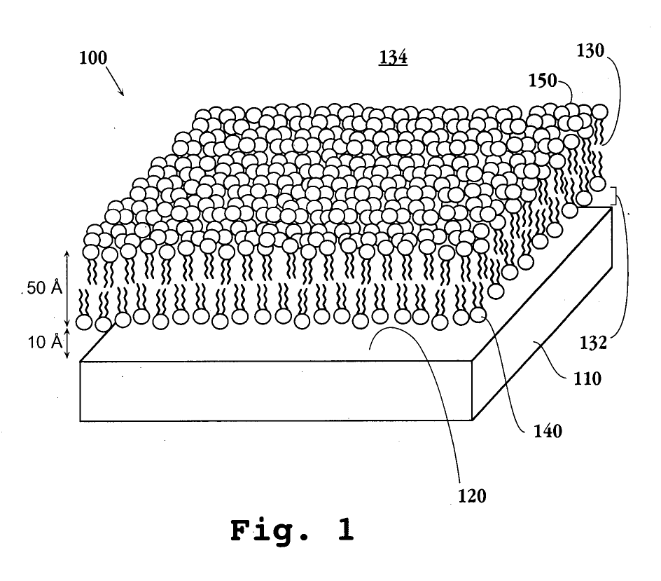

The term “supported lipid bilayer” refers to a planar bilayer of lipids or lipid-related materials on a solid support such as glass, mica, or oxidized polydimethylsiloxane (PDMS), such as shown in FIG. 1. The components of the supported bilayer are free to diffuse in the plane of the bilayer. Proteins, including integral membrane proteins, can be incorporated into the supported bilayer, but often they lose activity and/or lateral mobility.

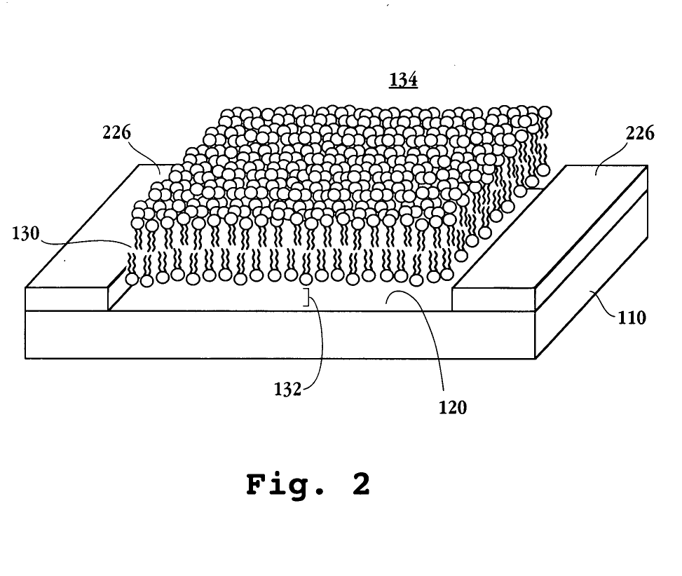

The term “patterned supported lipid bilayer”, such as is shown in FIG. 2, refers to a planar bilayer that is partitioned by patterning the surface using a variety of barrier materials, membrane blotting, microcontact printing or laminar flow.

The term “vesicle” refers to a roughly spherical, free-standing bilayer consisting of lipids or lipid-related materials. A vesicle is unilamellar if it contains a single bilayer or multilammellar if it contains several bilayers. A vesicle is typically a closed surface so that the vesicle contents and molecules outside the vesicle exchange slowly under ordinary conditions. Vesicles can be prepared by sonication of dispersions of lipid components in water or buffer or by extrusion of such solutions through membranes with defined pore sizes. Vesicles can be prepared with diameters from tens of nm to tens of mm. In addition to lipid content, the vesicle bilayer can contain proteins, glycolipids and other biological molecules that are typically associated with biological membranes. Because the environment is like that in a normal cell membrane, proteins are typically fully functional. The inside of the vesicle can be used to trap molecules providing a probe for the integrity of the vesicle enclosure, as sensors for changes in properties of the interior (e.g. pH, ion concentrations and the like) or for studies of content mixing upon vesicle fusion or rupture. An exemplary vesicle is shown in FIG. 3.

The term “tethered vesicle” refers to a vesicle that is connected to a surface by virtue of molecules on the surface of the vesicle interacting, either covalently or non-covalently, with molecules on the tethering surface. An exemplary tethering method is described below involving tethering a lipid vesicle to a supported lipid bilayer using oligonucleotide base pair complimentarity, a non-covalent but highly specific interaction whose strength can be varied by choice of the sequence using well-known algorithms developed to describe the stability of double helical oligonucleotides. In addition, the length of the tether, and hence distance of the tethered vesicle or bilayer from the supported bilayer surface, can be varied over a wide range by varying the length of the oligonucleotides involved in the formation of the tether.

The term “aqueous” refers to a water-based liquid medium.

The term “aqueous film” refers to a film of aqueous medium, typically about 5-20 angstroms, and preferably about 10 angstroms, between a substrate surface and a lipid-bilayer region.

A surface “expanse” of lipid-bilayer refers to a substantially uninterrupted planar expanse of lipid-bilayer on the surface of a substrate. The “expanse” may be partitioned into separated lipid-bilayer regions in accordance with the invention.

A “receptor” is a macromolecule capable of specifically interacting with a ligand molecule. In cells, receptors are typically associated with lipid bilayer membranes, such as the extracellular, Golgi or nuclear membranes. Receptors for incorporation into expanses of lipids in vitro (e.g., supported bilayers) may either be purified from cells, recombinantly expressed, or, in the case of small receptors, chemically synthesized.

A “transmembrane receptor” is an integral membrane protein that, when present in a cell membrane, transduces a binding event occurring on the extracellular side of the membrane into an intracellular signal.

“Members of a receptor protein family” refers to two or more proteins that are related in structure and/or function within or between organisms. Determining that proteins are “members of a receptor protein family” may be done using computerized algorithms known to persons of skill in the art to carry out, e.g., primary, secondary, tertiary, or quaternary structure alignments. Representative algorithms such as BLAST and VAST may be obtained from the Computational Biology Branch, National Center for Biotechnology Information, National Institutes of Health, 8600 Rockville Pike, Bethesda, Md. 20894 USA, and may be run directly from the National Center for Biotechnology Information website, http://www.ncbi.nlm.nih.gov.

A “ligand” is a molecule capable of specifically binding to a receptor. Binding of the ligand to the receptor is typically characterized by a high binding affinity, i.e., K (a)>10 (5), and can be detected either as a change in the receptor's function (e.g., the opening of an ion channel associated with or part of the receptor) or as a change in the immediate environment of the receptor (e.g., detection of binding by surface plasmon resonance). Ligands for incorporation into expanses of lipids in vitro (e.g., supported bilayers) may either be purified from cells, recombinantly expressed, or, in the case of small ligands, chemically synthesized.

Binding is “specific” if it results from a molecular interaction between a binding site on a receptor and a ligand or between two complementary oligonucleotides, rather than from “non-specific” sticking of a ligand to a receptor. In cases where the ligand binds the receptor in a reversible manner, specificity of binding can be confirmed by competing off labeled ligand with an excess of unlabeled ligand according to known methods. Non-specific interactions can be minimized by including an excess of a protein (e.g., BSA) that does not have binding sites for either the ligand or receptor.

A “fluid membrane” is a membrane having a native or native-like bilayer structure. As one of ordinary skill will recognize, some “fluid membranes” (i.e., those having high proportions of saturated lipids and/or sterols) may not have appreciable fluidity, yet nonetheless will be considered to be “fluid membranes” for purposes of the present invention.

“Assaying an interaction between a test agent and a composition” means determining whether the test agent interacts with the composition. “Assaying an interaction between a test agent and a composition” may be done by detecting interaction of a test agent with a composition using any method now known to one of skill in the art, or later developed, and is intended to encompass binding assays, such as direct binding and displacement assays, electrophysiological assays, metabolic assays, etc.

A “test agent” is intended to encompass all manner of organic, inorganic, biological and non-biological molecules that may be used in conjunction with the methods of the present invention.

II. Lipid Bilayer Array

FIG. 1 is a perspective view of a portion of a lipid bilayer array 100 in accordance with one aspect of the invention. The array is fabricated from a substrate 110, such as SiO₂, MgF₂, CaF₂, and mica, as well as a polymer film, such as a polyacrylamide or dextran film. SiO₂ is a particularly effective material for the formation of a bilayer-compatible surface region. The dimensions of the substrate are typically between about 0.1 cm to about 10 cm per side and about 0.01 mm to about 1 cm in thickness.

The substrate surface contains a plurality of distinct bilayer-compatible surface regions 120. A lipid bilayer expanse 130 is carried on each of the distinct bilayer-compatible surface regions 120. The lipid bilayer expanses may be comprised of a number of lipid molecules, including, but not limited to, phosphatidylcholine, phosphatidylethanolamine, phosphatidylserine, phosphatidic acid, phosphatidylinositol, phosphatidylglycerol, and/or sphingomyelin.

The lipid bilayer expanses have inner 140 and outer 150 bilayer surfaces. Interposed between each bilayer-compatible surface region 120 and corresponding lipid bilayer expanse 130 is an aqueous film 132 that is between about 5 Å and 15 Å, typically about 10 Å, in thickness. In certain configurations, separation of up to 1 micron can be achieved, as disclosed in U.S. Patent Application Publication 2003/0096418 A1, published May 22, 2003, which is incorporated herein by reference. Covering the lipid bilayer expanse 130 is a bulk aqueous phase 134.

FIG. 2 shows an embodiment of the invention that includes one or more bilayer barrier regions 226 separating distinct bilayer-compatible surface regions 120. The bilayer barrier regions may be depressed, flush, or elevated as shown at 226 in FIG. 2, with respect to the bilayer-compatible surface 120. The bilayer barrier surface region may be formed from any of a variety of materials having such bilayer barrier surface properties, including gold, positive photoresist, aluminum oxide, and indium tin oxide. In embodiments having elevated barriers, the height of the barrier may range from tens of Angstroms to several micrometers or more. The width of the barriers is typically between about 100 nm and about 250 μm. Preferably, the width is between about 1 μm and 100 μm. In an alternative embodiment, the discrete lipid bilayer expanses in associated surface regions are separated from one another by self-limiting lateral diffusion, without physical barriers between the expanses on the substrate surface.

In one embodiment, the device contains between about 10 and about 100 distinct bilayer-compatible surface regions. In another embodiment, the device contains at least about 2500 distinct bilayer-compatible surface regions. In yet another embodiment, the device contains at least about 25, 000 distinct bilayer-compatible surface regions. In still another embodiment, the device contains at least about 2.5 million distinct bilayer-compatible surface regions.

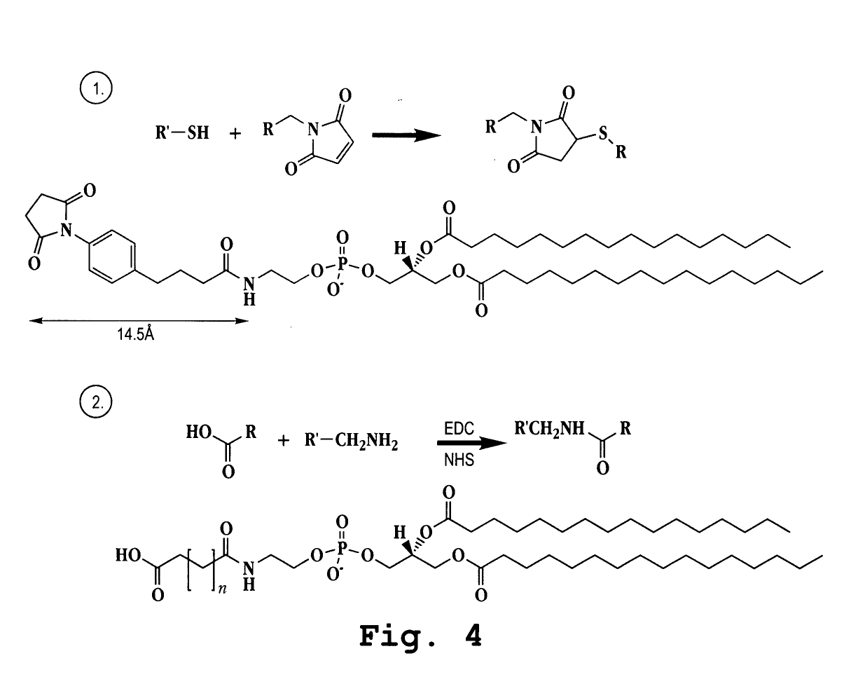

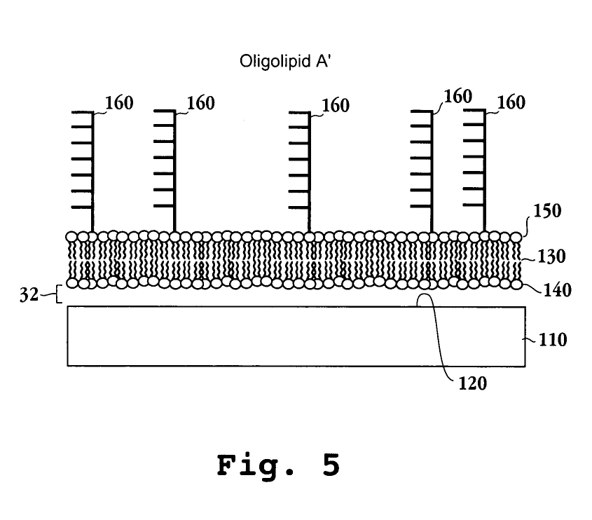

FIG. 5 is a view of an embodiment of the invention where the expanse 130 includes one or more lipids derivatized with an oligonucleotide 160 having a patch-specific oligonucleotide sequence and extending from the outer surface 150 of the associated expanse.

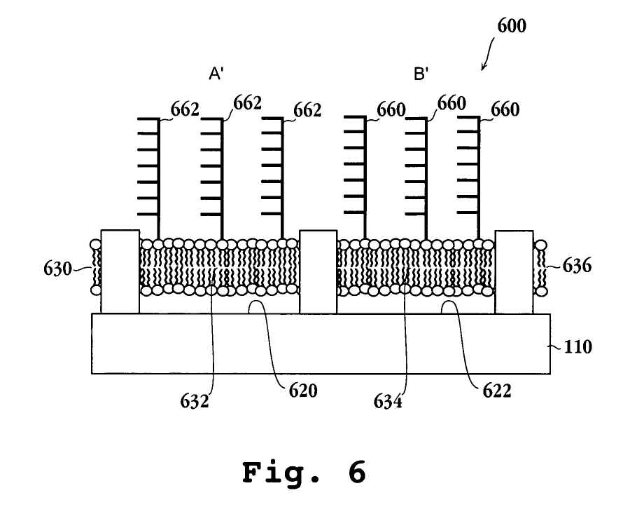

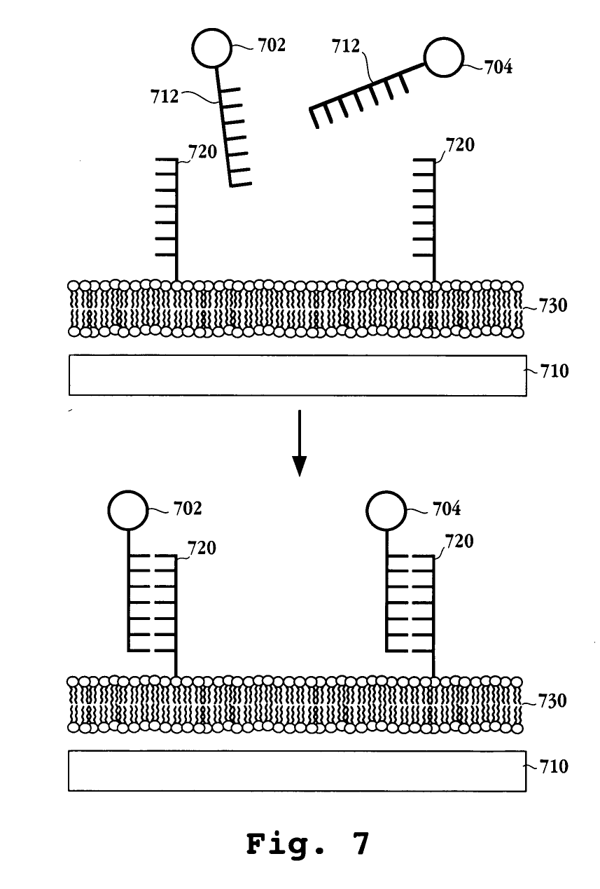

FIG. 6 shows an array 600 having a plurality of discrete lipid bilayer expanses 630, 632, 634 and 636 in associated surface regions 620 and 622 on support 110. Each outer bilayer expanse surface has patch-specific oligonucleotide sequences 660 and 662. In certain embodiments, at least one biomolecule such as a peptide, protein, carbohydrate, cytokine, growth factor, hormone, enzyme, toxin, drug, oligonucleotide, lipid, and combinations thereof, is anchored to at least one of the lipid bilayer expanses through a complementary oligonucleotide sequence capable of specifically hybridizing with the patch-specific oligonucleotide sequence in that expanse, such that the biomolecule is anchored to that expanse as shown in FIG. 7. Preferably, the biomolecule is a vesicle, receptor (such as a transmembrane receptor) or ion channel. FIG. 7 shows biomolecules 702 and 704, which can be the same or different, displaying oligonucleotide 710, whose sequence is complimentary to 720 which is displayed on the supported bilayer expanse outer surface 730, becoming tethered to surface 710 by sequence recognition and double helix formation. The tethered biomolecule is laterally mobile in the plane parallel to the bilayer expanse outer surface 730.

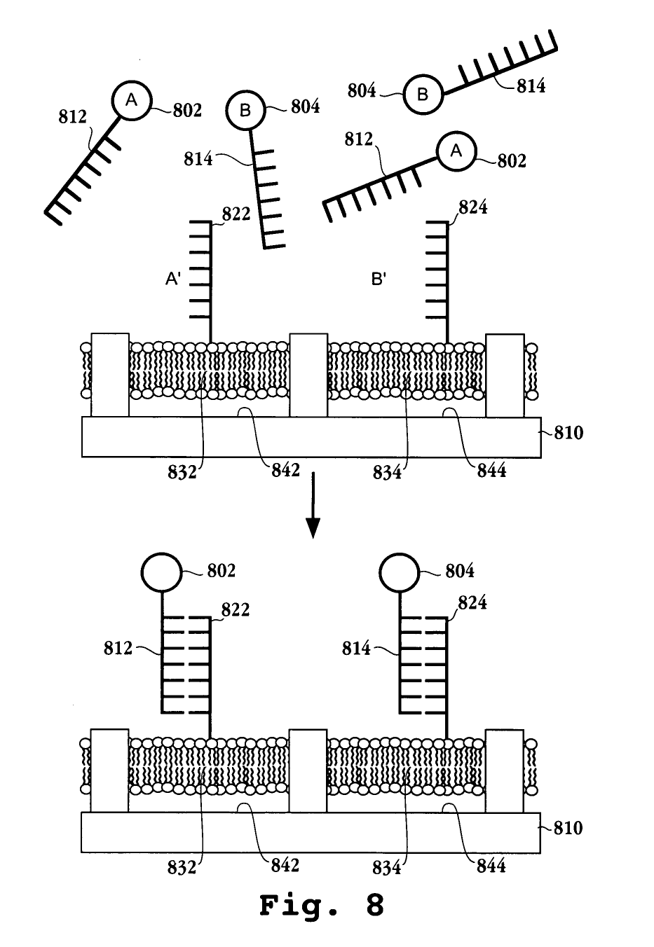

In other embodiments of the invention, as illustrated in FIG. 8, the array further includes one more discrete lipid bilayer patches associated with said expanses, where each patch contains a biomolecule anchored to the associated expanse through the hybridized oligonucleotides. Biomolecules 802, displaying oligonucleotides 812, whose sequence is complementary to patch-specific oligonucleotide 822 which is displayed on the supported bilayer expanse outer surface 832, become tethered to surface 810, in region 842, by sequence recognition and double helix formation. Biomolecules 804 displaying oligonucleotide 814, which has a sequence complementary to patch-specific oligonucleotide 824 which is displayed on the supported bilayer expanse outer surface 834, becomes tethered to surface 810 by sequence recognition and double helix formation. This places biomolecules, such as vesicles, whose contents are encoded by oligonucleotide 812 in corralled region 842, and concurrently places biomolecules whose contents are encoded by oligonucleotide 814 in corralled region 844. Thus, the lipid bilayer patches on different associated expanses may have the same or different compositions. As described, compositions may be encoded by the patch-specific oligonucleotide sequence in the expanse. The array may also include one or more second biomolecules associated with the bilayer patches, said second biomolecule(s) being able to move substantially freely within the associated patch. Preferably, the biomolecule corresponds to the oligonucleotide sequence, such that the identity of the biomolecule may be determined from the sequence of the oligonucleotide.

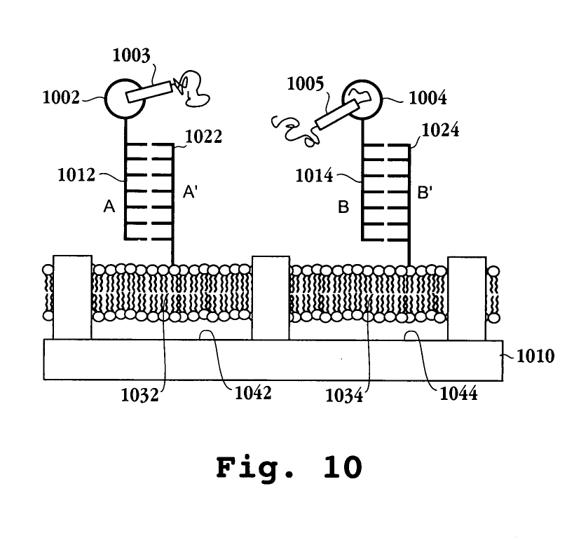

In a related embodiment, as illustrated in FIG. 10, the array further includes one more discrete lipid bilayer patches associated with said expanses, where each patch contains a biomolecule anchored to the associated expanse through the hybridized oligonucleotides. Biomolecule 1002, which includes a first membrane protein 1003, and displays oligonucleotides 1012, whose sequence is complementary to patch-specific oligonucleotide 1022 which is displayed on the supported bilayer expanse outer surface 1032, becomes tethered to surface 1010, in region 1042, by sequence recognition and double helix formation. Biomolecule 1004, which includes a second membrane protein 1005, and displays oligonucleotide 1014, which has a sequence complementary to patch-specific oligonucleotide 1024 which is displayed on the supported bilayer expanse outer surface 1034, becomes tethered to surface 1010 by sequence recognition and double helix formation. This places membrane protein 1003, whose contents are encoded by oligonucleotide 1012 in corralled region 1042, and concurrently places biomolecule 1005 whose contents are encoded by oligonucleotide 1014 in corralled region 1044.

When the barriers are removed, as illustrated in FIG. 11, interactions between biomolecules 1003 and 1005 can occur when tethered vesicles 1002 and 1004 diffusing on the surface encounter one another. This process can be enhanced or disrupted by agents in solution or associated with the vesicles.

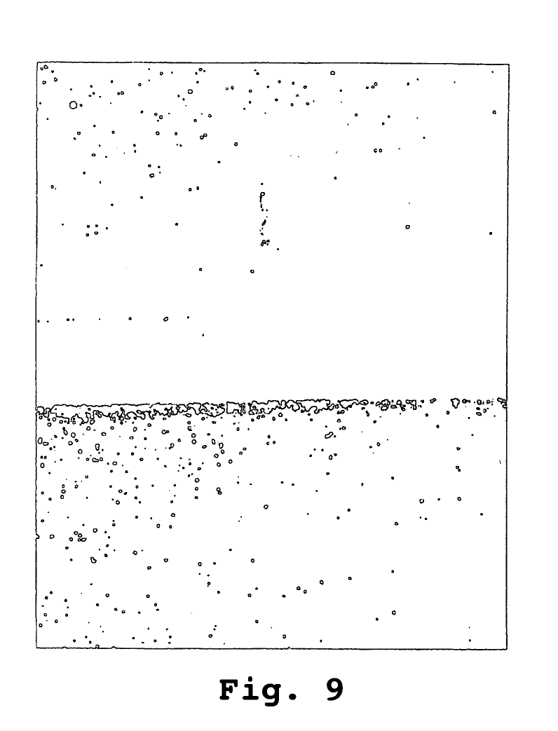

The lateral mobility of single vesicles tethered to a supported lipid bilayer is illustrated in FIG. 9. The tethered vesicles were labeled with a lipid bearing a fluorescent label for visualization. Individual spots are individual testhered vesicles. The image is partitioned by a barrier and the trajectories taken over time.

Tethered bilayers may be formed by oligonucleotide complementarity as shown in FIG. 12. In this embodiment, a large vesicle or vesicles displaying oligonucleotide 1212 and containing a specific biomolecule 1203 and/or lipid composition is bound to a supported lipid bilayer displaying the complementary oligonucleotide 1222. Upon rupture, for example by osmotic stress, a bilayer patch 1202 becomes tethered to the supported bilayer in region 1242. The distance of separation 1260 is determined by the length of the oligonucleotide sequences 1212 and 1222. FIG. 12 also illustrates the tethering of a different bilayer patch 1204, containing biomolecule 1205, to a separate region 1244 on a supported lipid bilayer outer surface 1234 by using the encoded vesicle tethering method described above, wherein tethered bilayer patch 1204 displays oligonuclotides 1014 which is complementary to oligonucleotides 1024 displayed on supported lipid bilayer outer surface 1234.

III. Method of the Invention

In one aspect of the invention, a method is described for associating or tethering lipid vesicles with a fluid supported bilayer membrane using mutual recognition elements. This association allows deposition of biomolecules, whether associated with the vesicle membrane or contained inside the vesicle, at specific locations on a surface. In this way, arrays of membrane-associated molecules can be displayed in a fully functional form in a controlled array format suitable for interrogation by a large number of different surface-sensitive techniques known to investigators skilled in this area. Because the tethered vesicles are themselves laterally mobile, vesicles containing different biomolecules such as proteins or glycolipids that may be involved in cell-cell, organelle-organelle and/or vesicle-organelle interactions can interact on the surface. Also disclosed are assays for such interactions and the ability to screen for molecules that enhance or disrupt these membrane-mediated interactions.

A. Synthesis of Oligolipids

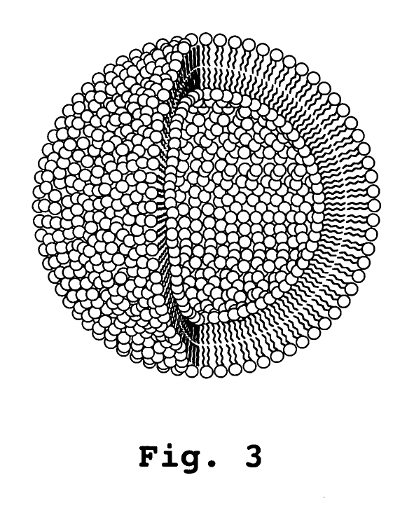

The lipid head group is modified or replaced by a DNA oligonucleotide or other molecule capable of sequence-specific recognition. The mechanism of attachment of the oligonucleotide involves chemistry well known to those of skill in the art of making modified oligonucleotides and modified lipids. The length of the oligonucleotide can range from a single base to many tens or hundreds of bases. Synthetic oligonucleotides are prepared by machine synthesis; much longer sequences can be be added by enzymatic ligation using methods well-known in the recombinant DNA field. Typical examples of linkages include covalent maleimide-SH coupling or amide bond formation as shown in FIG. 4, but many other attachment methods as are known to those of skill in the art may be used. Also, although the examples shown in FIG. 4 involve attachment to lipid head groups, the attachment could be to a simple fatty acid or a more complex lipid or other biomolecule. An exemplary preparation of oligonucleotide-modified vesicles is disclosed in Example 1A, below.

B. Preparation of Fluid Supported Bilayers Displaying Oligolipids

Supported lipid bilayers displaying one or more types of oligonucleotides are formed. The surface can display a single type of oligonucleotide, more than one type of oligonucleotide or the location of oligolipids on the surface can be controlled and corralled by membrane patterning techniques such as blotting, stamping and/or flow patterning as described below and in copending U.S. patent application Ser. No. 09/680,637, filed Oct. 6, 2000; Attorney Docket No. 58600-8204.US00, filed May 17, 2001; and U.S. Pat. No. 6,228,326, issued May 8, 2001, each of which is incorporated by reference herein in its entirety. Examples are shown in FIGS. 5 and 6. Because oligonucleotides are negatively charged, they exhibit no tendency to associate with the negatively charged glass surface. Furthermore, as with lipids themselves, the oligolipids are free to diffuse in the plane of the supported lipid bilayer membrane.

C. Sequence Encoded Vesicle Tethering

Vesicles displaying oligolipids on their surface are prepared by standard methods. If the sequence of the oligonucleotide displayed on the vesicle (the “sense” strand) is complementary to the sequence of the oligonucleotide displayed on the fluid supported surface (the “antisense” strand), then the vesicle becomes associated with the supported bilayer surface when flowed over that surface (FIG. 7). The lipid composition in the tethered vesicle can be the same or different from that in the supported bilayer. These tethered vesicles can be visualized by fluorescence microscopy using fluorescent labels in the vesicle lipid shell and/or the vesicle contents or by labeling other components (e.g. proteins) associated with the vesicles. Vesicles become associated with the supported bilayer when the sense and antisense oligolipids are present, that is, the tethering is sequence specific. Use of other non-complimentary sequences or blocking either the oligonucleotide displayed on the supported bilayer or vesicle surface by adding the antisense oligonucleotide, blocks vesicle tethering. These experiments support the concept that vesicle tethering by using this invention is highly specific and controllable.

Once associated with the supported membrane surface, the tethered vesicles are free to diffuse laterally in the plane of the supported bilayer. The positions of individual vesicles have been tracked by monitoring their fluorescence over time. Tethered vesicles diffuse with diffusion coefficients that are similar to the diffusion of lipids in the supported bilayer. Tethered vesicles can be moved in the plane by application of an electric field that is parallel to the plane of the supported bilayer surface. The direction of motion suggests that tethered vesicle motion is dominated by electroosmotic flow. When the electric field is turned off, the tethered vesicles relax back to a random distribution by diffusion. Tethered vesicles can also be moved in the plane by application of hydrodynamic flow. Such flow can be applied in any direction. When subject to flow on a supported bilayer surface that is patterned, the tethered vesicles are found to be confined and corralled by the supported bilayer barriers. These concepts are illustrated in FIGS. 7 and 8 as discussed above. A typical visualization of the tethered vesicles by fluorescence microscopy is shown in FIG. 9.

D. Assays Based on Contact Between Vesicles

Because the tethering process, in one embodiment of the invention, depends on complimentary recognition of sequence information on the vesicle and supported bilayer surfaces, and because oligolipids can be displayed on different parts of the surface, this invention allows for the spatial encoding of vesicles by their associated oligolipids such that specific vesicles can be targeted to specific regions on the surface. Because different specific vesicles can have different lipid, glycolipid, membrane protein or contents, this invention allows for targeted and specific display of vesicles on the surface. This is illustrated in FIG. 10 for two hypothetical integral membrane proteins (e.g. two different ion channels), as discussed above.

Because the tethered vesicles are laterally mobile, encounters between different vesicles, brought to specific locations on the surface by virtue of sequence complimentarity, can be monitored. If the tethered vesicles do not contain molecules that would lead to their interaction, they are observed to collide, but the collisions do not lead to sticking or mixing of tethered vesicle content, that is, they retain their integrity. However, if components are present that led to interactions, this leads to transient or permanent colocalization of the tethered membranes. In this way it is possible that vesicle-vesicle associations can be monitored in a planar format for individual vesicle pairs. For example, vesicle fusion processes are central to synaptic transmission in neurons and to trafficking of components within cells. Both processes involve intimate contact between protein and other factors displayed on the surface of the vesicle and its target cellular membrane. Factors that enhance or prevent such associations can be delivered to the surface in the planar format, providing a general method for assaying vesicle-mediated processes. This is illustrated in FIG. 11.

E. Advantages of the Present Invention

One advantage of the present invention is the ability to have integral membrane biomolecules distanced from the substrate such that they do not interact with the substrate which may cause denaturation of the biomolecule with loss of function or it may limit the lateral mobility of the membrane biomolecule, which is often important for function. Some investigators have described strategies for lifting the supported bilayer away from the underlying solid support by the use of polymer cushions or by some tethering strategies. In most cases, a substantial immobile fraction of proteins is observed and the activity of the proteins in such cushioned bilayers is often reduced. By working with vesicles, full function can be retained. This invention demonstrates a method for capturing the advantages of spatial organization and lateral mobility that is present in a supported bilayer, while retaining function as found in vesicles. The present method also allows for the encoding of information in the binding sequence by the use of oligonucleotides to produce diversity and afford unlimited lateral mobility.

F. Tethered Bilayers

Methods have been described for breaking open giant vesicles using osmotic stress in the presence of a supported lipid bilayer. Free-standing bilayer patches from the giant vesicle are found in close proximity to the supported bilayer, however, the interaction is unstable and this proximity cannot be controlled. By using the oligolipid tethering strategy described herein, it is possible to create and control bilayer patches separated from the supported bilayer by a distance that depends on the length of the oligolipids used. Furthermore, by using a patterned supported bilayer displaying different oligolipids created by methods known in the art and described in the applications and patents incorporated by reference above, it is possible to spatially encode the location of tethered membrane patches. This is illustrated in FIG. 12. The potential advantage of this organization is that the tethered bilayer is planar, in contrast to the spherical tethered vesicles described above. This geometry is especially useful for fluorescence microscopy and for surface sensitive techniques that monitor the contents of the region between the tethered and supported bilayer such as impedance spectroscopy.

G. Active Tethers

It may be desirable to be able to alter the tether in a specific temporal or spatial manner, for example, to allow release of the tether following the initiation of some process. Linkages can be constructed which are readily broken by addition of small molecules (e.g. a disulfide linkage can be broken by addition of a reducing agent) or by light induced fragmentation of the linkage. The former can be rendered spatially selective by addition under laminar flow conditions; the latter can be rendered spatially selective by scanning the illuminated region.

H. Spatially Selective Manipulation of Supported Lipid Bilayers

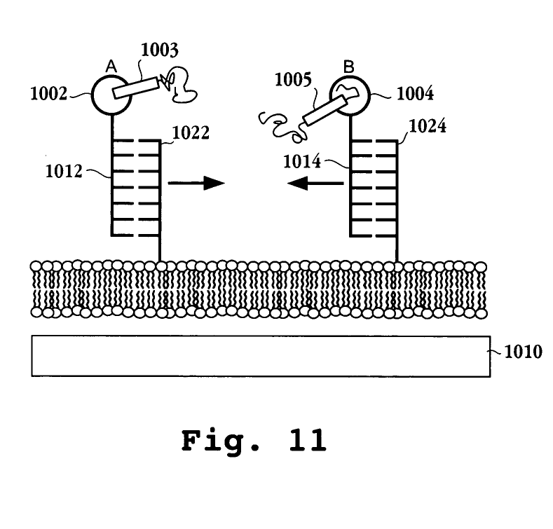

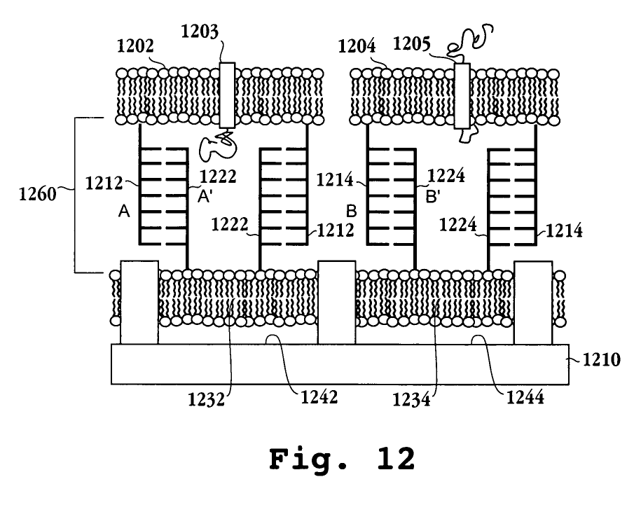

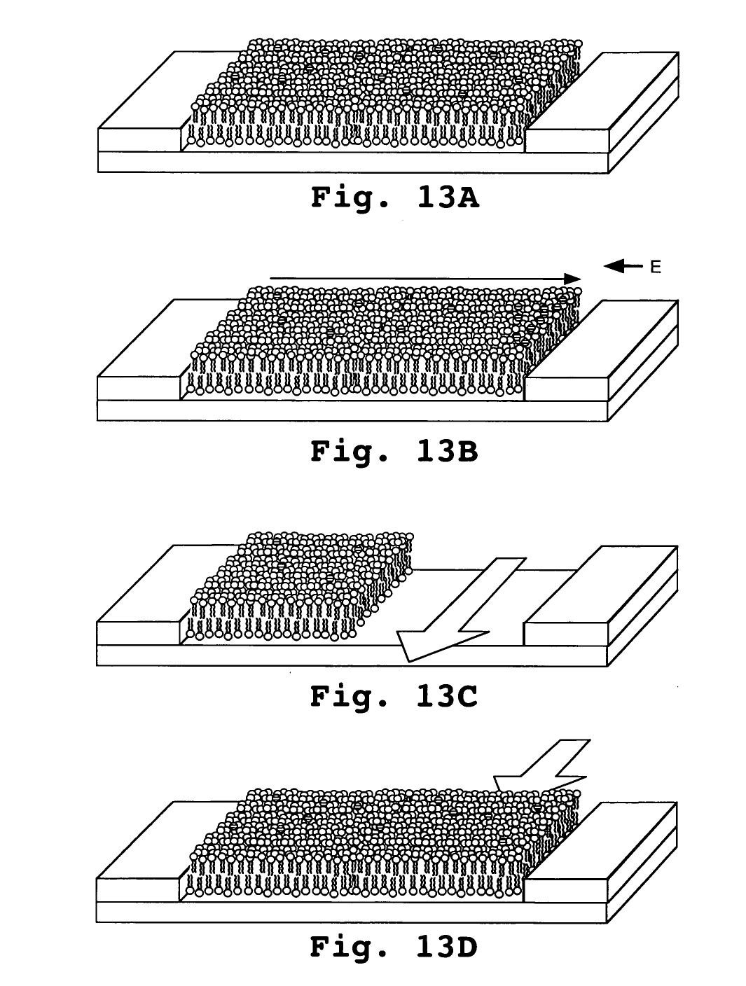

The present invention provides methods for using a laminar flow stream for removal of a bilayer from a specific area on a surface and, in some cases, replacement of this with a bilayer of a different composition or a barrier material. Directed removal/replacement of lipid bilayer can be achieved using mixing regions of extent much smaller than the size of the corrals. The basic experimental concept is illustrated in FIG. 13C and builds on the manipulations outlined in FIGS. 1A & B, in which charged (red) lipids have been accumulated against a micropatterned barrier using an electric field. Laminar flow concepts are used to direct a solution which strips the membrane from only a specified region as illustrated in FIG. 1C, completely and selectively removing the red component from the surface. This leaves behind a membrane edge, but because lateral expansion is self-limiting, the remaining bilayer does not substantially extend, or only extends slightly, into this newly exposed region. Using the stripping solution as described below, the remaining surface can be clean glass, similar to that produced by either scratching or blotting. If desired, the open region can be replaced with a bilayer of the same or different composition by flowing vesicles over the surface using the same inlet used to introduce the stripping the solution. These methods have applications in separations of membrane components, including mobile and immobile components on the surface, switchable (reversible) barriers, and surface analysis as outlined below.

In laminar flow, mixing between adjacent streamlines is normally diffusion limited. While posing a challenge in microfluidic design, this property has also been exploited in microscale patterning and chemical synthesis²³⁻²⁵. In the present invention, laminar flow is used to spatially confine the exposure of a bilayer to a stripping solution, allowing removal of a specified region of bilayer. In the most basic form, this process introduces a bilayer edge, which acts as a diffusion barrier. The functionality gained using laminar flow as described in the instant invention is the ability to both collect lipids from and reintroduce vesicles or other materials onto specific regions of a surface. These two concepts provide a number of new strategies for the manipulation, control and analysis of membrane composition.

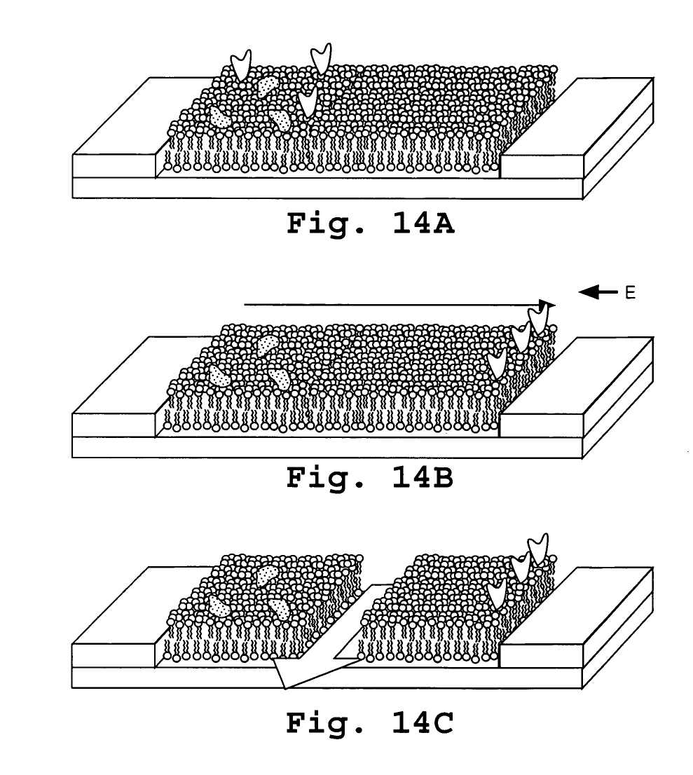

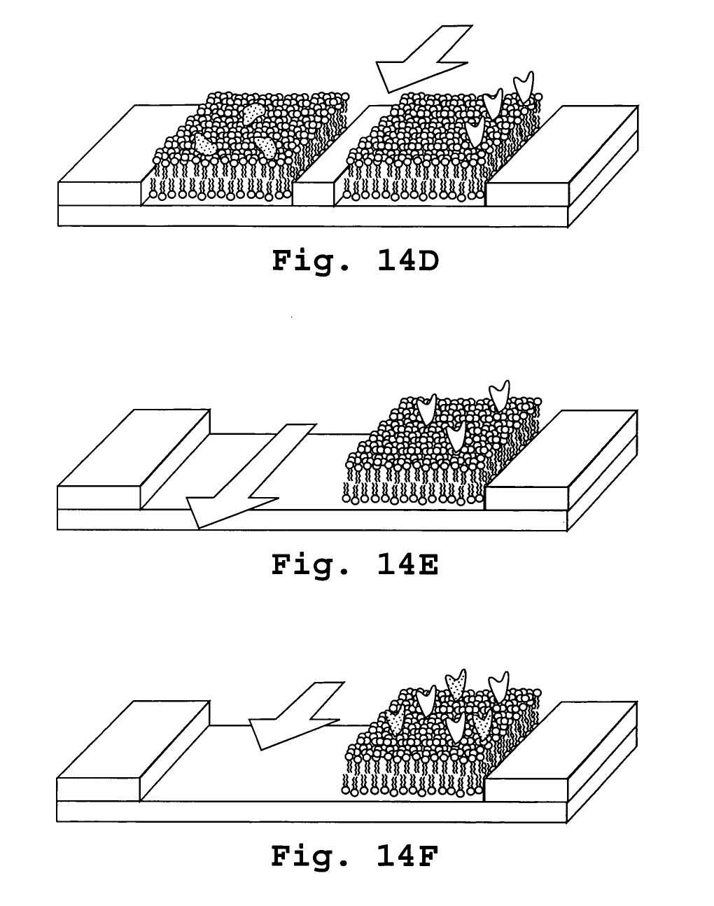

One application of supported bilayers is to display membrane associated proteins or other biomolecules in a planar format and under conditions that mimic their native environment. Lateral mobility is one aspect of such experiments, and the presence of an immobile fraction of membrane-associated protein, often a substantial fraction, complicates data interpretation. Several strategies have been developed to create supported membrane systems containing proteins^{10,26-25}. However, no general method has been described for directly producing bilayers containing large biomolecules, such as proteins, that are free of any immobile fraction. The present invention provides a solution to this issue, as illustrated schematically in FIG. 14. First, laminar flow of vesicles containing the protein of interest and vesicles with no protein is used to create two connected regions of bilayer. As illustrated, the region on the left of FIG. 14A contains a mixed population of both mobile and immobile target biomolecules represented by the “Y” shaped and membrane-embedded forms, respectively; these drawings are not intended to explain the origin(s) of mobile and immobile populations, but only distinguish the physical state of the two groups of biomolecules. The region on the right consists of bilayer alone. Under the influence of a driving force such as an electric field (FIG. 14B) or by diffusion alone, mobile biomolecules, but not the immobile ones, can move from one region to the other, previously unoccupied region. As a result, the membrane on the right side of FIG. 14B contains a purified population of mobile target biomolecules. The use of a driving force can result in collection of virtually all of the mobile protein in the new region. In itself, the establishment of this system by laminar flow addresses a fundamental challenge in the use of supported lipid bilayers; however, unless diffusion of the mobile population back to the region containing the immobile fraction is prevented, these two populations will ultimately remix. This can be accomplished using the membrane stripping method described here to remove a stripe of bilayer (FIG. 14C), which can be either left as open glass or filled in by flowing a barrier-forming material, such as protein, over the same region (FIG. 14D). Alternatively, the entire region containing immobile targets can be removed (FIG. 14E), allowing the introduction of pure lipids or additional, laterally mobile protein in order to increase the concentration of targets, or a different protein (as illustrated in FIG. 14F) to examine the interaction of these proteins with each other or with additional structures. These combinations make possible entirely new classes of membrane-based experiments, as pure populations of mobile proteins are available. More generally, these methods could be used in any membrane-based separation experiment to either remove a contaminating molecule or introduce new bilayer components into an established system.

The second broad class of applications is to use the stripping method to remove material from the surface for subsequent chemical analysis. Experiments such as the generation of gradients by electrophoresis, outlined in FIGS. 13A and B, encode fundamental properties of membrane biomolecules (e.g., mobility and intermolecular association) into lateral position. In one embodiment, fluorescence microscopy is used to determine the location and concentration of various components within the lipid bilayers. The fluorescent probes used to label these components are often of similar molecular weight to lipids and other membrane components. The influence of the additional size and physical or chemical properties of these probes as well as probe-probe interactions (e.g., fluorescence resonance energy transfer and quenching) on the behavior and quantitative characterization of bilayer components can be complex. For example, subtle interactions between membrane components leads to raft structures, and it would be very desirable to know the actual composition as a function of position if raft components are reorganized.

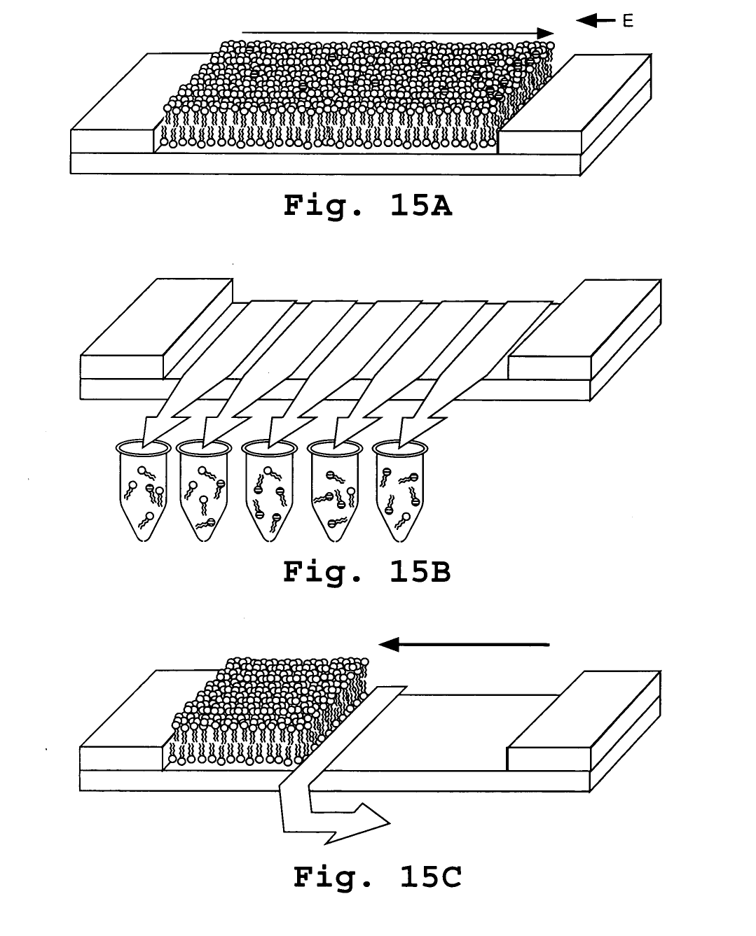

Alternatively, or in combination with the above methods, bilayer material may be collected from specific regions of the surface for off-line, label-free, high sensitivity analysis such as capillary electrophoresis, mass spectrometry or enzyme-based colorimetry. Two such evolutions of the present system are illustrated schematically in FIG. 15. One method involves the use of multiple flow streams to simultaneously remove and separate small strips of the bilayer (FIG. 15B), thereby sampling the distribution of components in the direction of the electrophoretic gradient. Alternatively, the stream can be scanned across the sample, by either moving the inlet relative to the sample or by controlling the flow rates of adjacent streams, for example (FIG. 15C). By using only one outlet, this approach facilitates a simplified sample handling and analysis strategy. The extent of the transitional edge, which is likely a result of detergent diffusion between the solution streams, may be reduced as desired. In the examples described below, the channel and corral dimensions were chosen to be on the order of hundreds of micrometers to facilitate manual alignment of flow and bilayer. However, the present invention contemplates the use of channel and corral dimensions less than 100 micrometers, preferably less than 10 micrometers, and more preferably less than 1 micrometer. Increased flow rates and modifications of solution properties are also contemplated by the present invention.

The methods demonstrated here take advantage of the unique fluid and surface-associated properties of lipid bilayers and form the foundation of biomembrane microfluidics. Experimental systems that are made possible using these concepts and are contemplated by the present invention include membrane-based separations, determination of protein-ligand or lipid-lipid association and dynamics, and analysis/directed assembly of membrane biomolecule complexes such as large protein assemblies and microdomains. Reintroduction of components to a bilayer is not limited to vesicles; other compounds, such as proteins, can be introduced onto a surface, leading to the dynamic formation of barriers based on these materials, such as illustrated in FIG. 14D. These methods may be easily integrated into existing microfluidic systems, leading to new devices for the preparation and analysis of biological membranes.

From the foregoing, it can be seen how various objects and features of the invention are met.

### III. EXAMPLES

The following examples further illustrate the invention described herein and are in no way intended to limit the scope of the invention.

### Example 1

### Spatially Encoded and Mobile Arrays of Tethered Lipids

A. Preparation of Oligonucleotide-Modified Vesicles

A mixture of lipids containing egg phosphatidylcholine and 0.5 mol % of reactive lipid, 1,2-dipalmitoyl-sn-glycero-3-phosphoethanolamine-N-[3-(2-pyridyldithio)propionate] (sodium salt) (N-PDP-PE, Avanti Polar Lipids), and fluorescently labeled lipid, Texas Red 1,2-dihexadecanoyl-sn-glycero-3-phosphoethanolamine (TR DHPE, Molecular Probes) in chloroform, is dried to a film, reconstituted in buffer (100 mM borate, 50 mM citrate, 100 mM NaCl, 2 mM EDTA, pH 8.0) to 25 mM and extruded through a 50 nm polycarbonate membrane (Avanti) to form vesicles. An oligonucleotide modified with a disulfide group on the 5′-end (IDT DNA technologies) is first reduced to expose a free sulfhydril functionality with 10 molar excess tris(2-carboxyethyl)phosphine (TCEP), then added to the vesicle solution to a final DNA concentration of 50 μM and lipid concentration of 13 mM. The DNA attaches to the outside surface by a disulfide exchange reaction on average 1-2 per vesicle, estimated using an assay based on the fluorescence of a labeled antisense oligonucleotide. Vesicles are isolated on a Sepharse CL-4B gel filtration column with the same buffer as eluant. Supported bilayers displaying oligonucleotides are formed on a cleaned glass coverslip by vesicle fusion as described earlier for simple lipids (Salafsky, J.; Groves, J. T.; Boxer, S. G. Biochemistry 1996, 35, 14773-81), and excess vesicles are rinsed away with copious amounts of buffer. Vesicles, at approximately 70 μM in lipids, displaying the complementary oligonucleotide, are incubated with this bilayer at room temperature for 30 min followed by further rinsing with buffer to remove free, unattached vesicles. Similar procedures were used to prepare vesicles varying in size from 30 to 200 nm and oligonucleotide lengths of 16 to 24 bases.

B. Visulation of Individual Tethered Vesicles and Their Mobility

Individual egg phosphatidylcholine vesicles containing 1% Texas Red DHPE and displaying the 24-mer sequence 5′-AGC GGA TM CM TTT CAC ACA GGA-3′ were tethered to the complementary sequence displayed on a supported lipid bilayer. The 2D diffusive motion of some vesicles is shown and is best visualized by video microscopy. See Yoshina-lshii, C. and Boxer, S. (2003) J. Am. Chem. Soc. 125:3696-3697, which is incorporated by reference herein. Mean-square displacement (MSD) versus time plots of four vesicles analyzed using unweighted internal averaging over all time pairs 17 and their least-squares fits up to the 15th time interval (lines through the data, T=25° C.) were plotted. The diffusion coefficient is given by D=MSD/4 tfor a random walk in two dimensions. Each vesicle was tracked for 100 time steps at 500 ms intervals. A histogram, not shown, of diffusion coefficients of 57 vesicles for a typical experiment using 100 nm vesicles labeled with 1% Texas Red DHPE attached to a supported bilayer had a mean value of D=0.9 μm²/s.

### Example 2

### Spatially Selective Manipulation of Supported Lipid Bilayers

A. Materials and Methods

### Vesicle Preparation

Stock solutions of small unilamellar vesicles (SUV's) were prepared by extrusion. Briefly, egg phosphatidylcholine (egg PC) (Avanti Polar Lipids, Alabaster, Ala., USA) was dried from chloroform in glass flasks, then desiccated under vacuum for at least 90 minutes. These lipids were reconstituted in deionized water at a concentration of 5 mg/ml, and then extruded through 50-nm pore size polycarbonate membranes (extruder and membranes from Avanti). For visualization and demonstration purposes, vesicles of egg PC were prepared containing various amounts of a negatively-charged, Texas Red®-labeled phospholipid (TR-PE, Texas Red® 1,2-dihexadecanoyl-sn-glycero-3-phosphoethanolamine; Molecular Probes, Eugene, Oreg., USA) and/or a neutral, NBD-labeled phospholipid (NBD-PC, 1-acyl-2-[12-[(7-nitro-2-1,3-benzoxadiazol-4-yl)amino]dodecanoyl]-sn-glycero-3-phosphocholine; Avanti). For use in bilayer formation, the stock vesicle solutions were diluted 1:1 in 10 mM HEPES/138 mM NaCl/5 mM KCl, pH 8.5 solution (HBS).

Flow Channel Preparation

Borosilicate glass coverslips (22 mm×40 mm, #1.5, VWR Scientific, Media, Pa., USA) were cleaned by immersion into Linbro 7×detergent (ICN Biomedicals, Inc., Aurora, Ohio, USA) diluted 1:3 (v/v) in deionized water, then baked at 450° C. for 4 hours. These working surfaces were stored in a covered jar at room temperature and used within one week in experiments. Laminar flow channel/electrophoresis structures, illustrated schematically in FIG. 2, were prepared from two layers of polydimethylsiloxane (PDMS; Sylgard 184; Dow Corning, Midland, Mich., USA) elastomer. The first layer was cut to define a converging flow system; the region downstream of the convergence measured 3 mm×12 mm with a PDMS layer thickness of 1 mm. A 2 mm×10 mm stripe intersects the middle of the flow channel to allow application of an electric field perpendicular to the direction of flow. Holes were cut through a second, 2 mm thick layer of PDMS, forming inlets and outlets to the underlying flow channel; vesicle, wash, and stripping solutions were introduced through tubing inserted into the inlets, while pipette tips filled with water provided electrical coupling between platinum wire electrodes and the electrophoresis channel. Prior to assembly of the complete channel, the layers of PDMS were oxidized in an air plasma for 20 seconds.

### Surface Micropatterning

For some experiments, the coverslips were patterned with barriers of fibronectin by microcontact printing ¹⁵²² prior to assembly of the flow channel. Briefly, PDMS elastomer stamps containing topological representations of an array of rectangular corrals, each corral measuring 450 μm×950 μm surrounded by a 50 μm-wide border, were oxidized in an air plasma then coated with 100 μg/ml of fibronectin (Sigma, St. Louis, Mo., USA) in 0.01 M 8 phosphate buffer (pH 7.3) for 15 minutes. The stamps were dried using nitrogen gas, and then placed in contact with a coverslip for 15 minutes; for the duration of this period, a 15 g weight was placed on each stamp. The micropatterned coverslips were sequentially rinsed in 0.01 M phosphate/140 mM NaCl (pH 7.3) buffer, 1% n-octyl-β-D-glucopyranoside in HBS, and water, and then dried under nitrogen. Adjacent rows of corrals were offset by 250 μm; with this geometry, different fractions of the corrals in adjacent rows could be exposed to the stripping solution in a single step.

### Bilayer Formation and Stripping

Bilayers were formed by first introducing vesicle solutions into the flow channels; after 30 seconds the channels were rinsed extensively with HBS. For steps involving electrophoresis, the channels were rinsed with water prior to application of the electric field. Stripping buffer (SB) consisted of 2% (w/v) n-octyl-β-D-glucopyranoside (Sigma) in HBS. To allow for both introduction of the stripping buffer under laminar flow conditions and subsequent rinsing with buffer without interruption of flow, a two-way, four-port valve was used in an injection-loop configuration to introduce 200 pl aliquots of SB into fully developed flows of HBS. New lipid bilayers were formed on the stripped regions by introducing vesicle solutions into the flow channel, then stopping flow so the vesicle solutions could interact with the surface for 30 seconds. Finally, the channel was rinsed with HBS. All rinsing and solution exchange steps (e.g., replacing buffer with water) were carried out using at least 2 ml of the appropriate solution. For steps carried out under laminar flow conditions, all solutions were introduced into the flow channels using a syringe pump. Unless otherwise stated, the combined flow through the channel for these experiments was 4 ml/min, corresponding to an average flow velocity in the channel of 22 mm/sec.

B. Spatially-Selective Removal of a Pre-Formed Lipid Bilayer

To demonstrate the use of laminar flow in removing a limited and specified region of a supported lipid membrane, a uniform, unpatterned bilayer was formed in a converging flow channel. For visualization purposes, but without loss of generality for other membrane-associated biomolecules, the bilayer contained 0.5 mol % of Texas Red-labeled lipid (TR-PE, red). Under laminar flow conditions (Reynolds number ˜1), half of the bilayer was exposed to a 200-μl aliquot of stripping solution; this solution was based on the detergent octylgluocopyranoside, which is widely used to disrupt cellular membranes. This process resulted in complete removal of lipid bilayer from one side of the surface, in this case, the right-hand side. Furthermore, it is also possible to remove a stripe of bilayer using a converging system of three or more streams (data not shown). As with other methods for removing bilayers from the surface such as scratching blotting¹⁹ and exposure to air²¹, the edge is stable after a rapid lateral expansion of a few percent of the bilayer area.

The region of the surface that was exposed by the stripping solution was able to support formation of a new bilayer that is continuous with the bilayer remaining on the surface. The open surface was exposed to vesicles of egg PC containing NBD-labeled lipids (NBD-PC, green), which formed a bilayer on the right side of the surface, but not the left; vesicles were introduced using the laminar flow configuration, but the flow was interrupted for 30 seconds to allow vesicle fusion before rinsing. Lipids from the Texas Red- and NBD containing regions freely mixed, as evidenced by a broad transition zone between the bilayer regions that became wider with time (data not shown). This demonstrates that the newly formed bilayer is continuous with the original lipid membrane, that is, the system is completely self-healing.

The directed removal of bilayer using laminar flow can be done with precision on the order of several microns. This can be seen in the higher-magnification image of the edge shown and a line profile of the fluorescence intensity across this region. The bilayer edge is a region measuring less than five microns in width. Over this region, the bilayer edge exhibits micro-scale structure in the plane of the membrane, which is similar to that observed at the edge of a self-limited, expanding region of bilayer and appears as a sharp but continuous decrease in average fluorescence in the line trace. There is also a transitional region, measuring about 20 pm in width to the left of the bilayer edge, which appears to contain randomly distributed defects measuring several micrometers or smaller in width. The density and size of these defects is reflected in the decrease in average fluorescence intensity of the bilayer near the edge. A 20-pm diameter photobleach spot, centered on the bilayer edge, recovered fully and appeared identical to that of neighboring, non-photobleached regions; the affected area still exhibited both defects visible by light microscopy and a gradual decrease in fluorescence intensity near the bilayer edge (data not shown), suggesting that the bilayer is a fluid, connected structure that is interrupted by defects. These defects are likely the result of a low concentration of detergent due to the limited diffusive mixing between streams. The stripping buffer used in this study contained 2% of octylgluocopyranoside, a concentration well above the critical micelle concentration for this detergent (0.5%-0.7%). Good results were obtained with detergent concentrations as low as 1%. Further reduction in detergent concentration, in conjunction with the strategies outlined in the specification as discussed above, is contemplated by the present invention, and may help reduce the extent of the transition region and reduce the presence of defects. The more gradual decrease in bilayer intensity towards the left of the trace in is associated with nonuniform illumination of the field of view and is also present in data collected for a homogenous bilayer.

C. Separation and Removal of Charged Bilayer Components

In this experiment, the interface between stripping and non-stripping solutions in a laminar flow chamber was aligned with a micropatterned array of offset lipid corrals. IT is centered on one of these 950 μm×450 μm lipid bilayer corrals containing both TR-PE and NBD-PC at 0.5 mol % and 2 mol %, respectively; this is the case shown schematically in FIG. 13A. Upon application of an electric field, the negatively charged TR-PE lipids (red), but not the neutral NBD-PC lipids (green), migrated to the right edge of the corral. The substrate was then exposed to a flow of stripping solution and buffer such that the flows crossed the width of the corrals, and the interface between the solutions fell across a series of offset corrals. This process resulted in removal of roughly ⅓ of the lipid bilayer area, which included the TR-PE that was accumulated against the right-hand barrier. Because of the staggered arrangement of corrals, different fractions of the lipid bilayer were removed from the corrals in neighboring rows. Simply moving the position of the flow relative to the surface can provide finer control over the placement of the removed area. In a subsequent step, vesicles of egg PC containing TR-PE alone (0.5 mol %) were introduced onto the right side of the substrate using the same laminar flow configuration. The formation of a homogeneous TR-PE-containing bilayer on the right of the corral as well as subsequent mixing of the TR-PE and NBD-PC lipids demonstrates one method for introducing new membrane biomolecules into a corralled bilayer system. Note that the stripping process can be aligned to remove any part of the corral, either a side, a stripe down the middle, or multiple stripes if multiple inlets are used, as discussed above. A trace amount of the fibronectin barriers was deposited at the interface between the stripping and buffer streams; this linear smear of deposited protein was only visible by light microscopy at long exposures (20 times longer than that used to image the barriers). Remarkably, this small amount of protein introduces a barrier to lipid diffusion that cannot be healed over by subsequent introduction of vesicles. Based on FRAP measurements, each region is a typical bilayer, but the regions remain separated. By rinsing the protein-micropatterned surfaces with a detergent solution prior to introduction of vesicles, this smearing of protein was eliminated, making possible the introduction of a new, connected region of bilayer. This issue can be avoided by using different materials that resist detergent solvation, such as photoresist or alumina, as the barrier material.

D. Composition Arrays by Barrier Formation

Removal of a region of lipid bilayer also introduces a barrier to lipid diffusion, because it creates a bilayer edge^{19,21}. In this section, a flow-generated barrier was used to produce essential elements of a composition array. A bilayer containing 0.5% TR-PE and 2% NBD-PC was formed on a surface containing an array of corrals inside a converging flow channel. An 80 V/cm electric field was applied for 1 hour to induce migration of the TR-PE to one side of each corral, similar to that illustrated in FIG. 13B. This surface was then exposed to a laminar flow of detergent-based stripping solution and buffer alone; the detergent was used to remove the region of lipid bilayer that did not contain the accumulation of TR-PE. As a result of the staggered arrangement of corrals in the array layout, the flow of stripping buffer removed different fractions of the corrals in adjacent rows. The TR-PE in each corral was accumulated against the right-hand barriers, followed by introduction of detergent over the left side of the surface thus creating an edge to the corralled lipid bilayer, which is indicated by the presence of NBD-PC lipids (green signal). Over time, the TR-PE (red) in each corral diffused, approaching a uniform distribution within each corral. Since an equal amount of TR-PE lipids was accumulated against the right hand barrier in each corral and these lipids were not removed by the stripping solution, the concentration of TR-PE in each corral, after relaxation of the gradient, reflects the area over which this lipid can diffuse, i.e, it is directly related to the fraction of the lipid corral removed by the stripping solution. Thus, the brighter TRPE signal in the upper corral, compared to the lower corral, is a result of the removal of a greater fraction of the lipids in the upper corral during the stripping process. Further experimental details are discuss in Kam, L. and Boxer, S (2003) Langmuir 19, 1624-1631, which is incorporated in its entirety herein.

Although the invention has been described with respect to particular embodiments, it will be apparent to those skilled in the art that various changes and modifications can be made without departing from the invention.

### REFERENCES

1) Bayerl, T. M.; Bloom, M. Biophys. J. 1990, 58, 357-362.

2) Johnson, S. J.; Bayerl, T. M.; McDermott, D. C.; Adam, G. W.; Rennie, A. R.; Thomas, R. K.; Sackmann, E. Biophys. J. 1991, 59, 289-294.

3) Koenig, B. W.; Kruger, S.; Orts, W. J.; Majkrzak, C. F.; Berk, N. F.; Silverton, J. V.; Gawrisch, K. Langmuir 1996, 12,1343-1350.

4) Schoenwaelder, S. M.; Burridge, K. Curr. Op. Cell Biol. 1999, 11, 274-286.

5) Giancotti, F. G.; Ruoslahti, E. Science 1999, 285, 1028-1032.

6) Viola, A.; Lanzavecchia, A. Apmis 1999, 107, 615-23.

7) Sackmann, E. Science 1996, 271, 43-8.

8) Watts, T. H.; McConnell, H. M. Ann. Rev. Immunol. 1987, 5, 461-75.

9) McConnell, H. M.; Watts, T. H.; Weis, R. M.; Brian, A. A. Biochim. Biophys. Acta 1986, 864, 95-106.

10) Grakoui, A.; Bromley, S. K.; Sumen, C.; Davis, M. M.; Shaw, A. S.; Allen, P. M.; Dustin, M. L. Science 1999, 285, 221-7.

11) Dori, Y.; Bianco-Peled, H.; Satija, S. K.; Fields, G. B.; McCarthy, J. B.; Tirrell, M. J. Biomed. Mat. Res. 2000, 50, 75-81.

12) Boxer, S. G. Curr. Op. Chem. Biol.; 2000, 4, 704-709.

13) Groves, J. T.; Boxer, S. G. Acc. Chem. Res.; 2002, 35, 149-157.

14) Groves, J. T.; Ulman, N.; Boxer, S. G. Science 1997, 275, 651-653.

15) Kung, L. A.; Kam, L.; Hovis, J. S.; Boxer, S. G. Langmuir 2000, 16,6773-6776.

16) van Oudenaarden, A.; Boxer, S. G. Science 1999, 285, 1046-1048.

17) Groves, J. T.; Boxer, S. G. Biophys. J. 1995, 69,1972-1975.

18) Cremer, P. S.; Groves, J. T.; Kung, L. A.; Boxer, S. G. Langmuir 1999, 15, 3893-3896.

19) Hovis, J. S.; Boxer, S. G. Langmuir 2000, 16, 894-897.

20) Hovis, J. S.; Boxer, S. G. Langmuir, 2001, 17, 3400-3405.

21) Cremer, P. S.; Boxer, S. G. J. Phys. Chem. B; 1999, 103, 2554-2559.

22) Kam, L.; Boxer, S. G. J. Am. Chem. Soc.; 2000,122,12901-12902.

23) Kenis, P. J. A.; Ismagilov, R. F.; Whitesides, G. M. Science; 1999, 285, 83-85.

24) Chiu, D. T.; Jeon, N. L.; Huang, S.; Kane, R. S.; Wargo, C. J.; Choi, I. S.; Ingber, D. E.; Whitesides, G. M. Proc Natl Acad Sci USA 2000, 97, 2408-13.

25) Delamarche, E.; Bernard, A.; Schmid, H.; Michel, B.; Biebuyck, H. Science 1997, 276, 779-81.

26) Wagner, M. L.; Tamm, L. K. Biophys. J.; 2001, 81, 266-275.

27) Wagner, M. L.; Tamm, L. K. Biophys. J.; 2000, 79, 1400-1414.

28) Groves, J. T.; Wulfing, C.; Boxer, S. G. Biophys. J. 1996, 71, 2716-2723.

29) Kam, L. C.; Perez, T. D.; Nelson, W. J.; Boxer, S. G. Biophys. J.; 2002, 82, 551A-551A.

It is claimed:

1. An array of separated lipid bilayers, comprising
a substrate having a surface defining a plurality of distinct bilayer-compatible surface regions,
a plurality of discrete lipid bilayer expanses in associated surface regions, said expanses having inner and outer bilayer surfaces,
an aqueous film interposed between each bilayer-compatible surface region and the lower surface of the corresponding lipid bilayer expanse,
each of said expanses containing one or more lipids derivatized with an oligonucleotide having a patch-specific oligonucleotide sequence and extending from the outer surface of the associated expanse,
a bulk aqueous phase covering the lipid bilayer expanses, and
at least one biomolecule anchored to at least one of the lipid bilayer expanses through a complementary oligonucleotide sequence capable of specifically hybridizing with the patch-specific oligonucleotide sequence in that expanse, such that the biomolecule is anchored to that expanse.

2. The array of claim 1, wherein the array further includes one more discrete lipid bilayer patches associated with said expanses, where each such patch contains such a biomolecule anchored to the associated expanse through said hybridized oligonucleotides.

3. The array of claim 2, wherein the lipid bilayer patches on different associated expanses have different compositions.

4. The array of claim 3, wherein the different compositions of each lipid bilayer patch are encoded by the patch-specific oligonucleotide sequence in the expanse.

5. The array of claim 2, wherein one or more of the lipid bilayer patch is a vesicle.

6. The array of claim 2, further comprising one or more second biomolecules associated with the bilayer patches, said second biomolecule(s) being able to move substantially freely within the associated patch.

7. The array of claim 6, wherein at least some of the different bilayer patches have different second biomolecules.

8. The array of claim 1, wherein the biomolecule corresponds to the oligonucleotide sequence, such that the identity of the biomolecule may be determined from the sequence of the oligonucleotide.

9. The array of claim 1, wherein said discrete lipid bilayer expanses in associated surface regions are separated by one or more barrier regions.

10. The array of claim 1, wherein said discrete lipid bilayer expanses in associated surface regions are separated from one another by self-limiting lateral diffusion, without physical barriers between the expanses on the substrate surface.

11. The array of claim 1, wherein said distinct bilayer-compatible surface regions on the substrate are formed from a material selected from the group consisting of SiO₂, MgF₂, CaF₂, and mica.

12. The array of claim 1, wherein the lipid bilayer expanses are comprised of at least one lipid selected from the group consisting of phosphatidylcholine, phosphatidylethanolamine, phosphatidylserine, phosphatidic acid, phosphatidylinositol, phosphatidylglycerol, and sphingomyelin.

13. A method of using a lipid patch array to detect membrane-bound biomolecular interactions, comprising
incubating the array of claim 6 under conditions effective to allow for the formation of biomolecular complexes between the second biomolecules, and
detecting any formed biomolecule complexes.

14. The method of claim 13 for screening for molecules that enhance or disrupt membrane-bound biomolecular interactions, further comprising
contacting the array, prior to or after said incubating, with one or more molecules under conditions which allow for the interaction of said molecules with said biomolecules or biomolecular complexes,
detecting any formed biomolecular complexes, and
comparing the results from the previous step to the results from the detecting step of claim 13 to determine whether the one or more molecules enhanced or disrupted membrane-bound biomolecular interactions.

15. The method of claim 13, wherein the degree of complex formation is quantitated.

16. The method of claim 13, wherein said biomolecules are selected from the group consisting of peptides, proteins, carbohydrates, cytokines, growth factors, hormones, enzymes, toxins, drugs, oligonucleotides, lipids, and combinations thereof.

17. The method of claim 13, wherein said molecules are selected from the group consisting of peptides, proteins, carbohydrates, cytokines, growth factors, hormones, enzymes, toxins, drugs, oligonucleotides, lipids, and combinations thereof.

18. A method of manipulating lipid-bilayer regions on a substrate, comprising
applying, to the array of claim 1, a controlled laminar-flow stream of an aqueous liquid, under flow conditions effective to remove a portion of the expanse in the path of said stream, wherein remaining portions of said expanse are substantially retained in their original position(s) on said region, adjacent exposed portion(s) of said region.

19. A method of manipulating lipid-bilayer regions on a substrate, comprising
applying, to a substrate having formed thereon, a discrete, defined-shaped lipid expanse which is confined to a corresponding defined-shaped region on the substrate and separated by an aqueous film, a controlled laminar-flow stream of an aqueous liquid, under flow conditions effective to remove a portion of the expanse in the path of said stream, wherein remaining portions of said expanse are substantially retained in their original position(s) on said region, adjacent exposed portion(s) of said region.

20. A system for manipulating lipid-bilayer regions on a substrate, said system comprising
the substrate having formed thereon, a discrete, defined-shaped lipid expanse which is confined to a corresponding defined-shaped region on the substrate and separated by an aqueous film,
a flow generating device capable of applying a controlled laminar-flow stream to the substrate, and
a composition capable of selectively removing at least a portion of the expanse.
